(19) United States
(12) Patent Application Publication
(10) Pub. No.: US 2014/0094821 A1
(43) Pub. Date: Apr. 3, 2014

(54) Impactor for Securing an Implant to a Bone Surface

(71) Applicant: DEPUY PRODUCTS, INC., Warsaw, IN (US)

(72) Inventors: Christel M. Wagner, Plymouth, IN (US); Scott M. Leestma, Columbia City, IN (US)

(73) Assignee: DePuy Products, Inc., Warsaw, IN (US)

(21) Appl. No.: 13/629,943

(22) Filed: Sep. 28, 2012

(51) Int. Cl. A61B 17/56

(52) U.S. Cl. 606/99

(57) ABSTRACT

An impactor for use in securing an orthopaedic implant within a bone surface includes a shaft having a first end adapted to be impacted by a tool. The impactor further includes a head having a first end and a second end, wherein the first end of the head is connected to the second end of the shaft and a second end of the head includes an impaction surface. The head further includes an anti-rotation projection extending from the impaction surface and adapted for insertion within a groove formed by the implant to prevent rotation of the head and shaft when the head is in contact with the implant.

### TECHNICAL FIELD

The present disclosure relates to a surgical instrument and, more particularly, to an impactor for use in securing an orthopaedic implant within a bone surface.

### BACKGROUND

During orthopaedic arthroplasty procedures, impactors may be used to drive implant components into the healthy bone that remains. The impactors include a first end that applies force to an implant component and a second, opposite or free end. After resection/preparation of the bone surface, cement or a similar material may be applied to the prepared bone surface and the implant component is attached to the prepared bone surface. Thereafter, the impactor is situated adjacent the implant component and a tool, such as a hammer or mallet, is used to hit the free end of the impactor, thereby applying force to the implant component via the first end of the impactor and driving the implant component into the resected bone surface. The free end of the impactor may be hit multiple times to fully seat the implant component.

### SUMMARY

According to an illustrative embodiment, an impactor for use in securing an orthopaedic implant within a bone surface comprises a shaft having a first end adapted to be impacted by a tool. The impactor further includes a head having a first end and a second end, wherein the first end of the head is connected to the second end of the shaft. A second end of the head includes an impaction surface and an anti-rotation projection extending from the impaction surface and adapted for insertion within a groove formed by the implant to prevent rotation of the head and shaft when the head is in contact with the implant.

In a further illustrative embodiment, an impactor for use in securing an orthopaedic implant within a bone surface includes a shaft having a first end adapted to be impacted by a tool and a second end for removably attaching at least one end piece. The impactor further includes an end piece in the form of an impactor head having a first end and a second end. The first end of the head is attached to the second end of the shaft and a second end of the head includes an impaction surface and an anti-rotation projection extending from the impaction surface and adapted for insertion within a groove formed by the implant to prevent rotation of the impactor head and the shaft when the head is in contact with the implant. The head further includes a cavity formed within the impaction surface and adapted to accommodate a projection extending from the implant.

In other illustrative embodiments, the projection is parallel to a longitudinal axis of the head and extends from an edge of the impaction surface.

In further illustrative embodiments, the head includes a cavity adapted to accept a projection extending outwardly from an implant.

In still other illustrative embodiments, the implant is a tibial component and the head includes impaction surfaces on opposing sides of the cavity that are adapted to contact medial and lateral bearing surfaces or a perimeter of a surface of the tibial component, the cavity is adapted to accept a spine of the tibial component, and the anti-rotation projection extends into a groove formed by a concave posterior edge of the tibial component.

In further illustrative embodiments, the impaction surface is formed on opposing sides of the cavity and is generally planar.

In other illustrative embodiments, the impaction surface is formed on opposing sides of the cavity and includes convex surfaces that are adapted to conform to concave bearings surfaces of the implant.

In other illustrative embodiments, the shaft includes a connector that allows for attachment of different end pieces.

In still other illustrative embodiments, the connector is formed by a lever arm that operates a latch finger having a latch at an end thereof that cooperates with a projection in each end piece to retain the end piece on the shaft.

In other illustrative embodiments, the impactor head is integrally and non-removably attached to the shaft.

In still further illustrative embodiments, the implant is a tibial component and the head includes impaction surfaces on opposing sides of the cavity that are adapted to contact medial and lateral bearing surfaces of the tibial component. The cavity is adapted to accept a spine of the tibial component and the anti-rotation projection extends into a groove formed by a concave posterior edge of the tibial component.

Other aspects and advantages of the present disclosure will become apparent upon consideration of the following drawings and detailed description, wherein similar structures have similar reference numbers.

### BRIEF DESCRIPTION OF THE DRAWINGS

The detailed description particularly refers to the following figures, in which:

FIG. 1 is a perspective view of an impactor head attached to an end of a tool, wherein the impactor head is used to aid in driving a tibial component into the tibia of a patient;

FIG. 2 is an exploded perspective view of the tool and impactor head of FIG. 1;

FIG. 3 is a bottom perspective view of an end of the impactor head shown in FIGS. 1 and 2 and depicting an anti-rotation feature and a cavity within the impactor head;

FIG. 4 is a perspective view of the tool of FIGS. 1 and 2 with the impactor head of FIGS. 1-3 attached thereto and depicting the tool and the impactor head as they are guided toward an implant component;

FIG. 5 is a perspective view of the tool and the impactor head of FIG. 4 after the tool has been guided toward the implant component and the impactor head is in contact with the implant component; and

FIG. 6 is a cross-sectional view taken generally along the lines 6-6 of FIG. 5 and depicting a spine of the implant component within the cavity and further depicting the anti-rotation feature of the impactor head disposed within a groove formed by a concave surface on a posterior side of the implant component.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout this disclosure in reference to both the orthopaedic implants described herein and a patient's natural anatomy. Such terms have well-understood meanings in both the study of anatomy and the field of orthopaedics. Use of such anatomical reference terms in the specification and claims is intended to be consistent with their well-understood meanings unless noted otherwise.

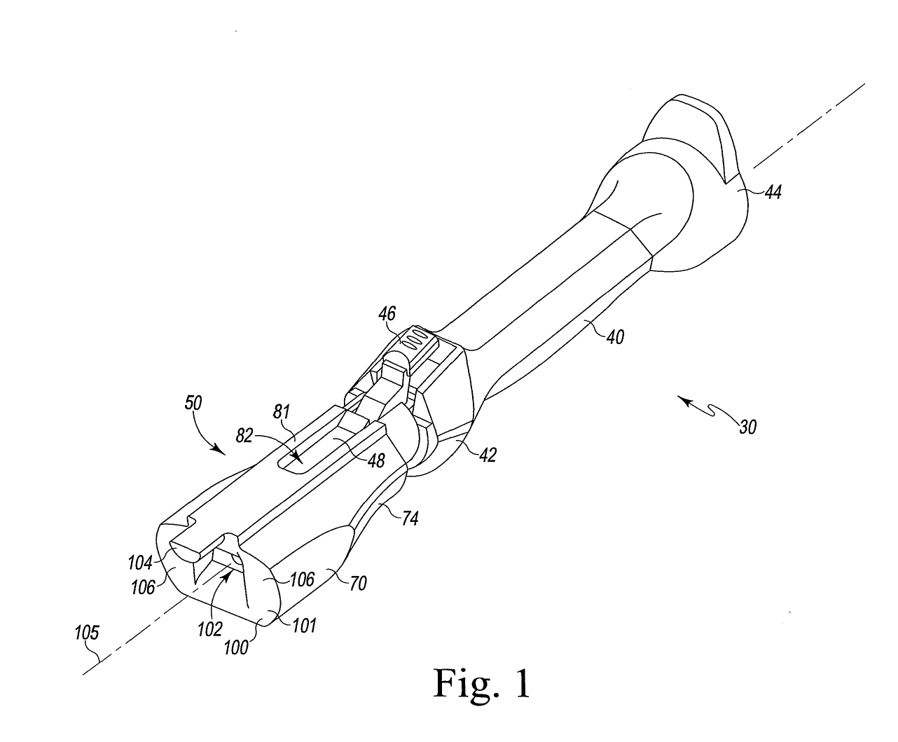

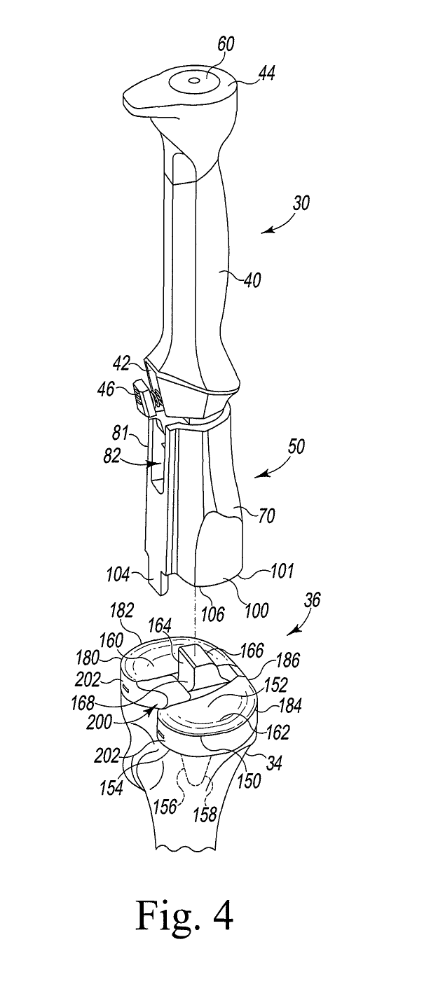

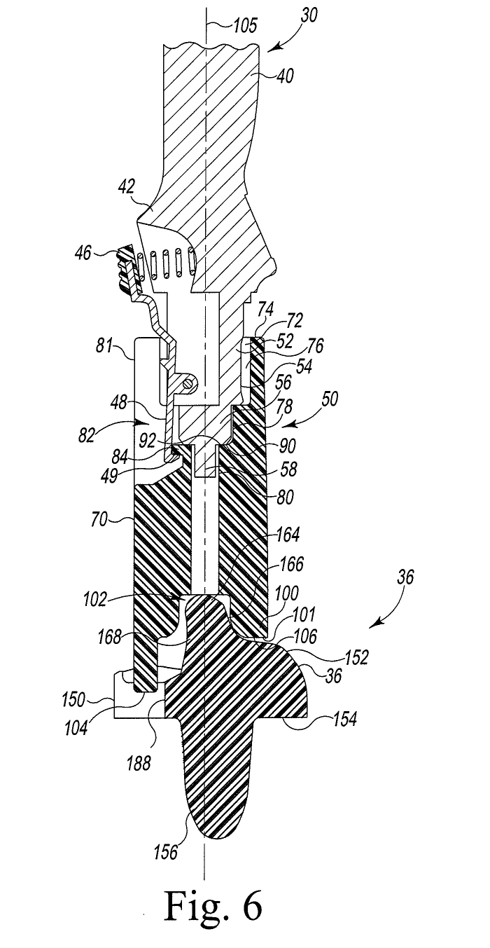

Referring now to FIGS. 1-3, a first embodiment of a tool 30 for driving an implant component (as shown in FIGS. 4-6) into a bone surface 34 is shown. The implant component is in the form of a tibial component 36 forming a portion of a posterior stabilized knee orthopaedic prosthesis in combination with a femoral component (not shown).

The tibial component 36 is illustratively formed from a polymeric material such as ultra-high molecular weight polyethylene (UHMWPE), but may be formed from other materials, such as a ceramic material, a metallic material, a bio-engineered material, or the like. The tibial component 36 is configured to be secured to the bone surface 34 in the form of a surgically-prepared proximal end of a patient's tibia and the femoral component is configured to articulate with the tibial component 36.

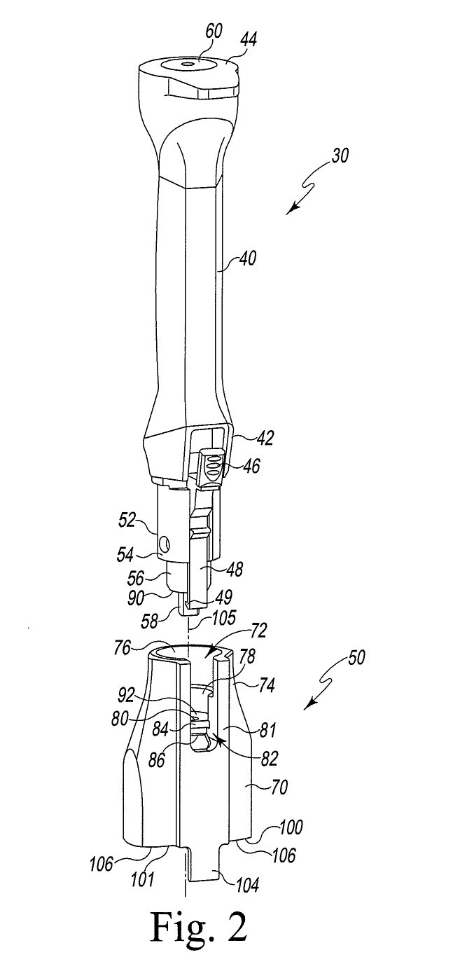

As best seen in FIGS. 1 and 2, the tool 30 includes a central shaft 40 having an attachment end 42 and a free end 44. The attachment end 42 includes a universal quick-release connector having a lever arm 46 that operates a latch finger 48. A latch 49 is disposed at an end of the latch finger 48 to easily attach and detach various instrument end pieces to the tool 30, for example, an impactor head 50. The latch finger 48 is biased to a closed position such that inward movement of the lever arm 46 opens the latch 49. The attachment end 42 further includes a central projection 52 formed of a plurality of concentric cylinders 54, 56, 58 each extending from the next and each having a smaller diameter than the cylinder 54, 56, 58 from which it extends, with the cylinder 58 having a smallest diameter and the cylinder 54 having a largest diameter.

While the tool 30 is described herein as being connected to an impactor head 50, the universal connector of the tool 30 may be connected to any instrument end piece including, but not limited to, punches, reamers, inserters, extractors, impactors, or any other instruments in which use of a hand piece or tool for controlling the instrument end piece is desired. During a particular surgery, the tool 30 may be utilized with a plurality of different end pieces to minimize the number of tools necessary in an operating room. Still optionally, the impactor head 50 may be integral with and non-removably attached to the tool 30.

The free end 44 of the tool 30 has a generally rounded form with a central, slightly raised circular portion 60, as seen in FIG. 2, that provides a strike zone for receiving a blow from a tool, such as a hammer or mallet, during use of the tool 30. The central shaft 40 forms a handle by which the tool 30 may be grasped during use.

Referring to FIG. 2, the impactor head 50 includes a body 70 having a cavity 72 formed in an attachment end 74 of the impactor head 50. The cavity 72 includes cylindrical walls 76, 78, 80 that are concentrically disposed with respect to one another and stepped in diameter to form corresponding cylindrical cavities. In particular, the wall 76 has a diameter greater than diameters of the walls 78, 80 and the wall 78 has a diameter greater than the diameter of the wall 80. The walls 76, 78, 80 are sized to accommodate the cylinders 54, 56, 58, respectively, of the central projection 52 extending from the attachment end 42 of the tool 30, as will be discussed in greater detail below.

Still referring to FIG. 2, the impactor head 50 further includes an outer wall 81 having a cutout 82 formed therein, wherein the cutout 82 is in partial communication with the cavity 72 and extends below the cavity 72. A protrusion 84 extends outwardly from a surface 86 formed in the cutout 82 and is spaced inwardly of the outer wall 81.

The impactor head 50 is illustratively formed from a polymeric material such as Radel® R polyphenylsulfone, but may be formed from other suitable materials.

The impactor head 50 is attached to the tool 30 by depressing the lever arm 46, thereby rotating the latch finger 48 about a hinge and lifting the latch finger 48. During this operation, the tool 30 is rotated until the latch finger 48 is aligned with the cutout 82 in the outer wall 80 of the impactor head 50 and the projection 52 of the tool 30 is inserted into the cavity 72 of the impactor head 50 with the cylinders 54, 56, 58 adjacent the walls 76, 78, 80, respectively. The projection 52 is inserted until an outer surface 90 of the cylinder 56 is adjacent an inner surface 92 forming a cylindrical cavity with the cylindrical wall 78. The lever arm 46 is thereafter released such that the latch finger 48 enters the cutout 82 and the latch 49 interferes with the protrusion 84 to prevent removal of the impactor head 50 from the tool 30. In a similar manner, the impactor head 50 may be removed by depressing the lever arm 46, thereby moving the latch finger 48 out of the cutout 82 and, thus, moving the latch 49 out of interference with the protrusion 84.

As discussed above, other instrument end pieces may be utilized with the tool 30. The other instrument end pieces may include structures similar to those of the impactor head 50, for example the cylindrical walls 76, 78, 80, the cutout 82, and the protrusion 84, to accommodate the attachment structures of the tool 30.

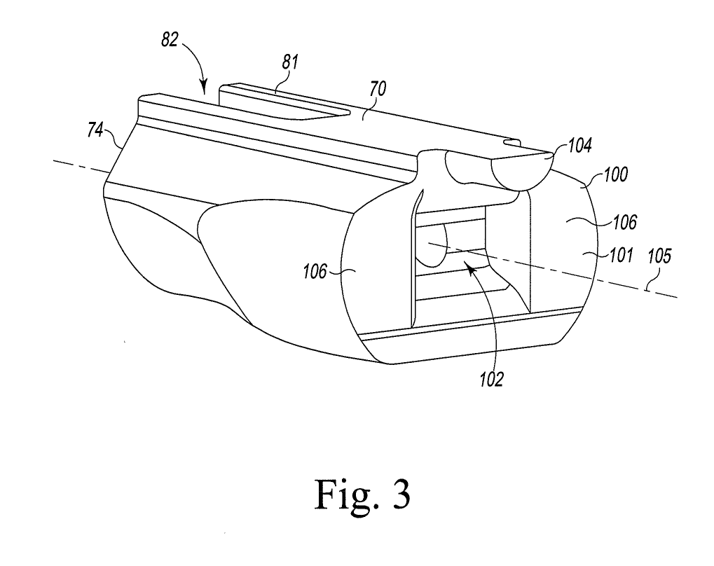

As best seen in FIG. 3, an impact end 100 of the impactor head 50 opposite the attachment end 74 includes a generally planar end wall 101 with a central cavity 102 formed in the planar end wall 101. In addition, an anti-rotation feature 104 extends outwardly and away from the planar end wall 101 adjacent the outer wall 81 of the impactor head 50. In particular, the anti-rotation feature 104 extends parallel to a longitudinal axis 105 of the impactor head 50. While the anti-rotation feature 104 is shown as a protrusion or projection, the anti-rotation feature 104 may take the form of any other structure that is sufficient to prevent rotation of the tool 30 and the impactor head 50.

Still referring to FIG. 3, the planar end wall 101 of the impactor head 50 includes impaction surfaces 106 disposed on opposing sides of the cavity 102, as will be discussed in greater detail below. While the end wall 101 is described as being planar, the end wall 101 may optionally have a different shape, for example, a shape that conforms to a shape of a tibial component 36 or other implant component with which the impactor head 50 is used.

As best seen in FIGS. 4-6, the tibial component 36 includes a platform 150 having an upper bearing surface 152 and a bottom surface 154. The tibial component 36 includes a stem 156 extending downwardly from the bottom surface 154 of the platform 150. The stem 156 is received into a bore 158 formed within the resected bone surface 34 of the tibia. The tibial component 36 is configured to be implanted within the bone surface 34, as will be discussed in detail below.

The upper bearing surface 152 of the tibial component 36 includes a medial bearing surface 160, a lateral bearing surface 162, and a spine 164 extending upwardly from the platform 150. The medial and lateral bearing surfaces 160, 162 are configured to receive or otherwise contact corresponding medial and lateral condyles of a femoral component (not shown). In this manner, the bearing surfaces 160, 162 may each have a concave contour. The spine 164 is positioned between the bearing surfaces 160, 162 and includes an anterior side 166 and a posterior side 168. The spine 164 is configured to be received within an intracondyle notch or recess formed between the medial and lateral condyles of the femoral component (not shown).

Still referring to FIGS. 4-6, the tibial component 36 further includes an outer edge 180 with convex medial and lateral edges 182, 184, a convex anterior edge 186, and a concave posterior edge 188. The shape of the outer edge 180 may generally conform to a shape of an outer surface of the resected tibia 34.

Although specific steps during the surgical procedure will be described in detail, one skilled in the art will appreciate that the present specification is not limited to these specific steps and the tool 30 with the impactor head 50 attached may be used in any sequence of steps as preferred by a particular surgeon and depending on the steps necessary during the surgical procedure.

Use of the tool 30 with the attached impactor head 50 during a total knee replacement surgical procedure will now be briefly discussed with reference to FIGS. 4-6. After resection and reaming of the tibia, drilling of the proper holes in the patient's tibia 34, and appropriate sizing of the tibial components using trial components, the tool 30 and the impactor head 50 are used to secure the tibial component 36 to the patient's resected tibia 34. Bone cement may be used to help secure the tibial component 36 in place.

The impactor head 50 is attached to the tool 30 and the combination is used to secure the tibial component 36 within the resected tibia 34. In particular, once the tibial component 36 is positioned appropriately within the resected tibia 34, the impactor head 50 is moved toward the tibial component 36, as depicted in FIG. 4, such that the anti-rotation feature 104 of the impactor head 50 is aligned with the concave edge 188 of the tibial component 36 and the cavity 102 of the impactor head 50 is aligned with the spine 164.

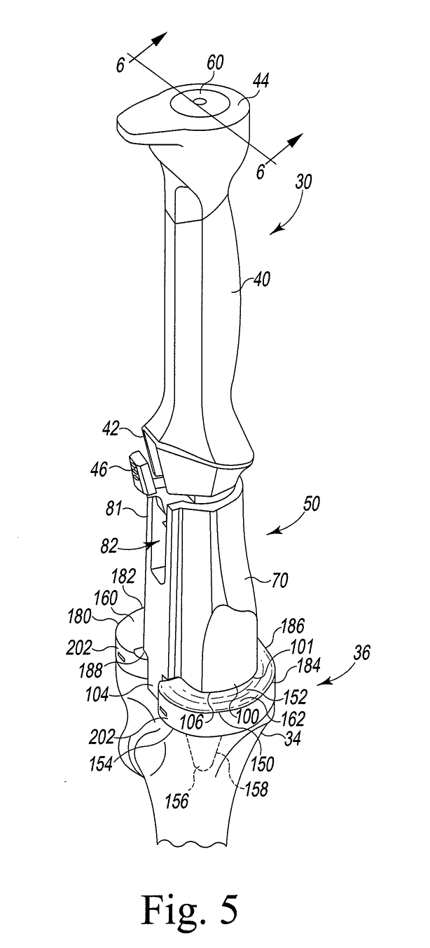

As depicted in FIG. 5, the impactor head 50 is positioned against the tibial component 36 with the impaction surfaces 106 of the impactor head 50 disposed adjacent the medial and lateral bearing surfaces 160, 162 of the tibial component 36. In this position, the cavity 102 allows the impaction surfaces 106 of the impactor head 50 to be disposed adjacent the bearing surfaces 160, 162 of the tibial component 36 by accommodating the spine 164 therein. In addition, when the anti-rotation feature 104 is positioned within a groove 200 formed by the concave edge 188, convex posterior edges 202 of the tibial component 36 prevent rotation of the impactor head 50.

After the impactor head 50 is positioned appropriately against the tibial component 36, the tibial component 36 is driven into the resected tibia 34 by using a hammer, mallet, or other suitable tool to strike the free end 44 of the tool 30. The force exerted by striking the free end 44 of the tool 30 drives the tibial component 36 into the bore 158 formed within the resected bone surface 34 of the tibia until the bottom surface 154 of the tibial component 36 is correctly seated against an upper resected surface 34 of the tibia. A surgeon may need to strike the free end 44 of the tool 30 multiple times to appropriately seat the tibial component 36.

The cavity 102 in combination with the spine 164 and/or the anti-rotation feature 104 in combination with the groove 200 allow for proper alignment of the impactor head 50 adjacent the tibial component 36 prior to and during use of the tool 30 and the impactor head 50. In this manner, proper alignment of the impactor head 50 with the tibial component 36 is facilitated and rotation of the tool 30 and the impactor head 50 is prevented. Forces transmitted by the tool 30 and the impactor head 50 will therefore be transmitted evenly and to the proper areas of the tibial component 36.

While a particular tibial implant component is depicted herein, the principles of the present application may be used with any tibial implant component. For example, the implant component of the present application may alternatively be in the form of a tibial component forming a portion of a cruciate retaining knee orthopaedic prosthesis in combination with a femoral component. Such a tibial component does not include a spine 164 and therefore, may be used with an impactor head 50 having the cavity 102 or may alternatively be used with an impactor head 50 that does not include the cavity 102. In a further embodiment, the tibial implant component may include a tibial tray and/or may include one or more pegs or other means for fixing the tibial implant component to a prepared tibial bone surface.

As will become apparent from reading the present specification, any of the features of any of the embodiments disclosed herein may be incorporated within any of the other embodiments without departing from the scope of the present disclosure.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. An impactor for use in securing an orthopaedic implant within a bone surface, the impactor comprising:
a shaft having a first end adapted to be impacted by a tool; and
a head having a first end and a second end, wherein the first end of the head is connected to the second end of the shaft and a second end of the head includes an impaction surface and an anti-rotation projection extending from the impaction surface and adapted for insertion within a groove formed by the implant to prevent rotation of the head and shaft when the head is in contact with the implant.

2. The impactor of claim 1, wherein the projection is parallel to a longitudinal axis of the head and extends from an edge of the impaction surface.

3. The impactor of claim 2, wherein the head further includes a cavity adapted to accept a projection extending outwardly from the implant.

4. The impactor of claim 3, wherein the implant is a tibial component and the head includes impaction surfaces on opposing sides of the cavity that are adapted to contact medial and lateral bearing surfaces or a perimeter of a surface of the tibial component, the cavity is adapted to accept a spine of the tibial component, and the anti-rotation projection extends into a groove formed by a concave posterior edge of the tibial component.

5. The impactor of claim 3, wherein the impaction surface is formed on opposing sides of the cavity and is generally planar.

6. The impactor of claim 3, wherein the impaction surface is formed on opposing sides of the cavity and includes convex surfaces that are adapted to conform to concave bearing surfaces of the implant.

7. The impactor of claim 2, wherein the shaft includes a connector that allows for attachment of different end pieces.

8. The impactor of claim 7, wherein the connector is formed by a lever arm that operates a latch finger having a latch at an end thereof that cooperates with a projection in each end piece to retain the end piece on the shaft.

9. The impactor of claim 2, wherein the impactor head is integrally and non-removably attached to the shaft.

10. An impactor for use in securing an orthopaedic implant within a bone surface, the impactor comprising:
a shaft having a first end adapted to be impacted by a tool and a second end for removably attaching at least one end piece; and
an end piece in the form of an impactor head having a first end and a second end, wherein the first end of the head is attached to the second end of the shaft and a second end of the head includes an impaction surface, an anti-rotation projection extending from the impaction surface and adapted for insertion within a groove formed by the implant to prevent rotation of the impactor head and the shaft when the head is in contact with the implant, and a cavity formed within the impaction surface and adapted to accommodate a projection extending from the implant.

11. The impactor of claim 10, wherein the second end of the shaft includes a connector that allows for attachment of different end pieces.

12. The impactor of claim 11, wherein the connector is formed by a lever arm that operates a latch finger having a latch at an end thereof that cooperates with a projection in each end piece to retain the end piece on the shaft.

13. The impactor of claim 10, wherein the projection is parallel to a longitudinal axis of the head and extends outwardly from an edge of the impaction surface.

14. The impactor of claim 13, wherein the implant is a tibial component and the head includes impaction surfaces on opposing sides of the cavity that are adapted to contact medial and lateral bearing surfaces of the tibial component, the cavity is adapted to accept a spine of the tibial component, and the anti-rotation projection extends into a groove formed by a concave posterior edge of the tibial component.

15. The impactor of claim 13, wherein the impaction surface is formed on opposing sides of the cavity and is generally planar.

16. The impactor of claim 14, wherein the impaction surface is formed on opposing sides of the cavity and includes convex surfaces that are adapted to conform to concave bearing surfaces of the implant.
